(19) United States
(12) Patent Application Publication
(10) Pub. No.: US 2005/0287146 A1
(43) Pub. Date: Dec. 29, 2005

(54) Method of inhibiting Candida-related infections using donor selected or donor stimulated immunoglobulin compositions

(76) Inventors: Joseph Patti, Cumming, GA (US); John Vernachio, Canton, GA (US); Yule Liu, Alpharetta, GA (US); Magnus Hook, Houston, TX (US); Maria Bowden, College Station, TX (US); Jenny K. Singvall, College Station, TX (US)

Correspondence Address: STITES & HARBISON PLLC, 1199 NORTH FAIRFAX STREET, SUITE 900, ALEXANDRIA, VA, 22314, US

(21) Appl. No.: 11/016,564

(22) Filed: Dec. 20, 2004

Related U.S. Application Data
(60) Provisional application No. 60/566,082, filed on Apr. 29, 2004.
(60) Provisional application No. 60/561,540, filed on Apr. 13, 2004.
(60) Provisional application No. 60/530,654, filed on Dec. 19, 2003.

(51) Int. Cl. A61K 39/395

(52) U.S. Cl. 424/151.1

(57) ABSTRACT

A method for treating or preventing infections from yeast of the Candida species is provided wherein an immunoglobulin composition containing high titers of antibodies to staphylococcal adhesins ClfA and SdrG is administered in an amount effective to inhibit the growth and progression of Candidial infections. The compositions and methods of the present invention are advantageous in that they can be used to treat both staphylococcal and Candidial infections at the same time, and they are particularly effective in treating or preventing late-onset sepsis in neonates.

### CROSS-REFERENCE TO RELATED APPLICATIONS

This application claims the benefit of U.S. Provisional Applications Ser. No. 60/566,082, filed Apr. 29, 2004, Ser. No. 60/561,540, filed Apr. 13, 2004, and Ser. No. 60/530,654, filed Dec. 19, 2003, all of said applications incorporated herein by reference.

### FIELD OF THE INVENTION

The present invention relates to the use of an immunoglobulin product obtained from purified donor plasma containing high antibody titers to MSCRAMM proteins ClfA and SdrG in the prevention and treatment of infections from Candida yeast, including Candida species late-onset sepsis and other Candida systemic infections.

### BACKGROUND OF THE INVENTION

Low birth weight (LBW) infants comprise 1.4% of all births in the United States, and over 57,000 infants per year are very low birth weight (VLBW) defined as <1,500 gm.¹ Advances in medical care provided by neonatal intensive care units (NICUs) throughout the country have dramatically improved the survival for these premature infants. One of the costs of prolonged survival among premature infants is an increased frequency of complications, especially nosocomial (hospital acquired) infections. In one study, the overall rate of late-onset infection among VLBW infants (“VLBWI”) 501 to 1,500 gm was 16% but the rate increased with decreasing birth weight and gestational age, rising rapidly to 40% among the smallest infants (500 to 600 gm).²

In a study of infants 401 to 1,500 gm birth weight admitted to the National Institute of Child Health and Human Development (NICHD) Neonatal Research Network centers, the rate of infection among infants of birth weight 401 to 1500 g after day three of life was 21%, essentially unchanged from previous estimates.³ In fact, late-onset sepsis has become the most common cause of death among premature infants after the third day of life.⁴

Reasons for the increased risk of infection among neonates include iatrogenic factors such as the use of vascular catheters, but also host factors. Immunoglobulin G (IgG), a critical part of immunity against bacterial pathogens, is transferred from mother to infant selectively through the placenta beginning at 8 to 10 weeks of gestation and accelerating during the last trimester. Infants born prior to 32 weeks gestation are relatively deficient in IgG. In vitro studies have demonstrated the importance of IgG directed against staphylococci for host defense.⁵⁻⁷ Low levels of IgG at birth is an identified risk factor for late-onset sepsis in LBW infants.²

The predominant organism for late-onset sepsis in recent studies is S. epidermidis and similar species collectively referred to as coagulase-negative staphylococci (CoNS).^{2, 8-10} While the mortality attributed specifically to CoNS is considered to be less than that of other organisms, the public health implications of CoNS infections are significant. Widespread use of antibiotics, especially vancomycin, for the treatment of suspected or proven nosocomial infection applies selective pressure for the emergence of antibiotic-resistant bacteria in intensive care units. Stoll et al. remarked “It is alarming that 44% of infants in this cohort (whether or not they had documented CoNS infection) were treated with vancomycin.”³ Vancomycin resistant strains of CoNS have been rarely reported, but the possibility of wider emergence of such strains would be disastrous.^{11, 12}

In addition to staphylococcal late-onset sepsis, fungal sepsis in VLBWI is significant medical problem. In a prospective study by Conner et al., among 1,111 VLBWI, 5% developed fungal sepsis within the first 28 days of life. The predominant fungal pathogen was Candida species of yeast (82%). In a similar study, the mortality rate associated with Candida species late-onset sepsis in VLBWI was 43.9%. However, despite the very severe pathological conditions caused by Candida-related infections, there have been very few effective treatment regimens against these extremely dangerous infections. Even further, it has not heretofore been possible to develop a treatment regimen which can address both infections caused by staphylococcal organisms and at the same time be effective against Candida-related infections.

It is therefore imperative that new strategies be developed which can address the critical problem of hospital-acquired infections in premature infants, and in particular, it is highly desirable to develop treatments and compositions which can be useful in treating and preventing Candida-related infections and at the same time be useful in inhibiting the progression of staphylococcal infections.

### SUMMARY OF THE INVENTION

It is thus an object of the present invention to provide compositions and methods for diagnosing, treating, and/or preventing infections caused by Candida species of yeast.

It is thus another object of the present invention to provide compositions and methods which are particularly useful in fighting late-onset sepsis in neonates and which can inhibit the growth and severity of infections caused by Candida species of yeast and staphylococcal infections at the same time.

It is still further an object of the present invention to provide donor selected or donor stimulated immunoglobulin compositions that can be effective in identifying and isolating surface antigens from Candida albicans and which can be useful in treating or preventing Candida-related diseases.

These and other objects are provided by the present invention wherein a donor immunoglobulin composition having high titers of antibodies to the proteins ClfA from S. aureus and SdrG from S. epidermidis can be administered to a patient in need of treatment for or protection against an infection caused by yeast of the species Candida such as Candidiasis, and this composition will be effective in inhibiting the yeast and enabling the effective treatment or prevention of the Candida infection. In addition, in another embodiment of the invention, an immunoglobulin composition of the invention can be prepared which includes a high titer to antigen from a Candida species yeast such as Candida albicans, and this composition can also be used effectively to inhibit Candidial yeast and thus treat or prevent a Candidial infection. Further, because of its ability to recognize surface proteins in Candida, the immunoglobulin compositions of the present invention will also be useful in identifying and isolating surface proteins from Candida yeast and in diagnosing Candida infections. The present compositions and methods will thus be particularly effective in treating or preventing late-onset sepsis in low birth weight neonates.

These and other objects of the present invention are obtained through the compositions and methods as set forth in the detailed description of the invention provided hereinbelow.

### BRIEF DESCRIPTION OF THE DRAWING FIGURES

FIG. 1 is a histogram showing staining of Candida albicans with decreasing concentrations of an immunoglobulin composition in accordance with the present invention.

FIG. 2 shows a Western Blot analysis of binding to antigens from a cell wall extract of Candida albicans to an immunoglobulin composition in accordance with the present invention

FIG. 3 is a graphic representation of the increased survival of mice receiving the immunoglobulin composition of the present invention following challenge with Candida albicans.

FIG. 4 shows the amino acid sequences of the ligand-binding regions of several bacterial adhesins.

FIG. 5 shows the amino acid sequences of the ligand-binding regions of several bacterial adhesins as compared with the N-terminal region of Als5 and Als7 of Candida albicans.

FIG. 6 shows the amino acid sequences of the Als proteins of Candida albicans.

FIG. 7 is a structural view of certain bacterial adhesins as compared with Als5 and Als7 of Candida albicans.

FIG. 8 is a photomicrograph showing that the Veronate® and Aurexis® compositions in accordance with the invention can recognize an Als protein from Candida albicans.

FIG. 9 is a schematic representation of 2-D Western blotting tests whereby the immunoglobulin compositions of the invention identify surface antigens from Candida albicans in accordance with the present invention.

FIG. 10 is a photomicrograph showing that the immunoglobulin compositions in accordance with the invention bind to and can identify surface antigens from Candida albicans.

FIG. 11 shows the results of chromatographic tests showing that the immunoglobulin composition in accordance with the invention recognizes Als3 protein from Candida albicans.

FIG. 12 shows a summary of the surface antigens from Candida albicans which can be identified using the immunoglobulin composition in accordance with the present invention.

### DETAILED DESCRIPTION OF THE PREFERRED EMBODIMENTS

In accordance with the present invention, there is provided a method for inhibiting infections caused by Candidial yeast, and this method comprises administering an effective amount of a purified human donor plasma containing a higher than normal antibody titer to the adhesin ClfA (clumping factor A) from Staphylococcus aureus and a higher than normal antibody titer to the adhesin SdrG, an Sdr (serine-aspartate repeat) protein from Staphylococcus epidermidis. This donor plasma can be obtained using either the selection approach or the stimulation approach as set forth as disclosed in U.S. Pat. No. 6,692,739, incorporated herein by reference. The use of either method results in immunoglobulin compositions that have an antibody titer to each of the selected adhesins in an amount that is higher than that found in pooled intravenous immunoglobulin obtained from unselected donors. As disclosed in U.S. Pat. No. 6,692,739, the desired immunoglobulin compositions can be obtained through the selection of donors identified as having high titers to the desired adhesins of interest or through the stimulation of donors by vaccination with the desired adhesin or adhesins. In accordance with the present invention, these donor immunoglobulin compositions have now unexpectedly have been discovered to recognize surface proteins from Candida species of yeast and can thus be used in methods of inhibiting, diagnosing, treating or preventing infection from Candidial yeast, as set forth in more detail below.

As indicated above, the present method comprises the administration of an effective amount of a donor immunoglobulin composition as described above to a patient in need thereof so as to inhibit, treat or prevent an infection from a Candida yeast such as Candida albicans. By effective amount, as would be recognized by one skilled in the art, is meant that amount which will be effective in inhibiting infection from Candida yeast so as to treat or prevent a condition caused by this yeast species, and one would readily recognize that this amount will vary greatly depending on the nature of the infection and the condition of a patient. Accordingly, an “effective amount” of the immunoglobulin compositions in accordance with the invention generally comprises a nontoxic but sufficient amount of the composition or effective agent therein such that the desired prophylactic or therapeutic effect is produced. Thus, the exact amount of the immunoglobulin composition that is required will vary from subject to subject, depending on the species, age, and general condition of the subject, the severity of the condition being treated, the particular carrier or adjuvant if being used, its mode of administration, and the like. Accordingly, the “effective amount” of any particular donor composition will vary based on the particular circumstances, and an appropriate effective amount may be determined in each case of application by one of ordinary skill in the art using only routine skills. The exact amount administered to patients will thus be adjusted to suit the individual to whom the composition is administered and will vary with age, weight and metabolism of the individual. The compositions may additionally contain stabilizers or pharmaceutically acceptable preservatives, such as thimerosal(ethyl(2-mercaptobenzoate-S)mercury sodium salt) (Sigma Chemical Company, St. Louis, Mo.).

In accordance with the invention, purified donor immunoglobulin compositions are obtained in accordance with any of the methods described in U.S. Pat. No. 6,692,739, and such compositions will contain a higher than normal antibody titer to the Clumping Factor A (ClfA) protein from S. aureus, as further described, e.g., in U.S. Pat. Nos. 6,008,341 and 6,177,084, incorporated herein by reference, and the serine-aspartate dipeptide repeat G (SdrG) protein from S. epidermidis as described in more detail in U.S. Pat. No. 6,635,473, incorporated herein by reference. One such formulation is INH-A21 which has been obtained from donor-selected staphylococcal human immune globulin and which has previously been shown to be useful for the prevention and treatment of staphylococcal infections in low birth weight (LBW) and very low birth weight (VLBW) infants. In the preferred embodiment, prior to administration to patients in order to inhibit, treat or prevent a Candidial infection, INH-A21 is preferably nanofiltered, and solvent-detergent treated to remove and inactivate viruses. It is generally desired that formulations in accordance with the invention such as INH-A21 contain at least 5% IgG in a suitable sodium chloride concentration and preferably does not contain sucrose or preservatives. In a preferred form, INH-A21 contains IgG, 5% weight per volume of which >95% is monomer. Other classes of immune globulin are present in low amounts, with IgM <0.5% and IgA <0.5%. The range of IgG subclass composition is similar to that found in normal human plasma and is within the following ranges: IgG₁, 50-75%; IgG₂, 20-40%; IgG₃, 1-10%; and IgG₄ less than 5%.

In the particularly preferred embodiment, plasma used in the production of INH-A21 is derived from donors specifically selected for elevated levels of antibodies against the staphylococcal fibrinogen binding proteins, SdrG and ClfA. These surface expressed molecules belong to a family of proteins called Microbial Surface Components Recognizing Adhesive Matrix Molecules (MSCRAMM®). SdrG and ClfA are found on the surface of >95% of strains of Staphylococcus epidermidis and Staphylococcus aureus, respectively. These antigens play an important role in the adherence of bacteria to host tissues, which is the initiating step in establishment of infection. It has now been demonstrated that in addition to higher titers of antibodies to the staphylococcal adhesins described above, the composition INH-A21 in accordance with the present invention also contains elevated levels of antibodies to antigens expressed by Candida species. This is a very important scientific finding with significant clinical ramifications, since Candida infections are often difficult to detect and treat. Accordingly, formulations in accordance with the present invention such as INH-A21 can be useful for the prevention and treatment of Candida infections, particularly in low and very-low birth weight infants. As indicated above, in the preferred method of the invention, the method comprises administering to a patient in need thereof, such as a low or very-low birth weight infant, a purified immunoglobulin composition with high titers of antibodies to ClfA and SdrG, and this composition is administered in an amount and for a time effective to achieve the therapeutic treatment goals, namely inhibition, treatment or prevention of a Candidial infection.

In another embodiment of the present invention, plasma used in the production of a purified immunoglobulin composition may be prepared by screening for donors which have a high titer against an antigen from a Candida yeast such as Candida albicans, and preparing a purified immunoglobulin composition in the manner set forth above which in this case will have an antibody titer to an antigen from Candida which is higher than that which is found in pooled intravenous immunoglobulin obtained from unselected donors. In particular, immunoglobulin compositions may be provided in accordance with the invention which have a higher than normal titer to a surface antigen from a Candida yeast such as Candida albicans. In accordance with the present invention, these antigens can be selected from the group consisting of the Als proteins, enolase, INO1-myo-inositol phosphate synthase, glucose-6-phosphate dehydrogenase, methionine synthase, ADH1/alcohol dehydrogenase; FBA1/fructose-biphosphate aldolase and the homologue of S. cerevisiae HEM13 which is involved in HEME synthesis. As described above, the preferred method of treating patients such as low and very-low birth weight infants is by administering to a patient in need thereof the purified immunoglobulin composition with high titers of antibodies to Candida antigens in an amount and for a time effective to achieve the therapeutic treatment goals, namely inhibition, treatment or prevention of a Candidial infection.

In light of the fact that the immunoglobulin compositions of the invention can recognize a series of Candidial antigens, in another embodiment of the present invention, a method of diagnosing a Candidial infection is provided which comprises introducing an immunoglobulin composition having an antibody titer to an S. aureus Clumping Factor A (ClfA) protein in combination with an antibody titer to an S. epidermidis serine-aspartate repeat G (SdrG) protein in an amount higher that that found in pooled intravenous immunoglobulin obtained from unselected donors to a sample suspected of containing antigens from Candida yeast for a time sufficient to allow the Candida antigens to bind to the antibodies in the immunoglobulin composition, and diagnosing a Candidial infection by determining if Candida antigens in the sample have bound to the antibodies in the immunoglobulin composition.

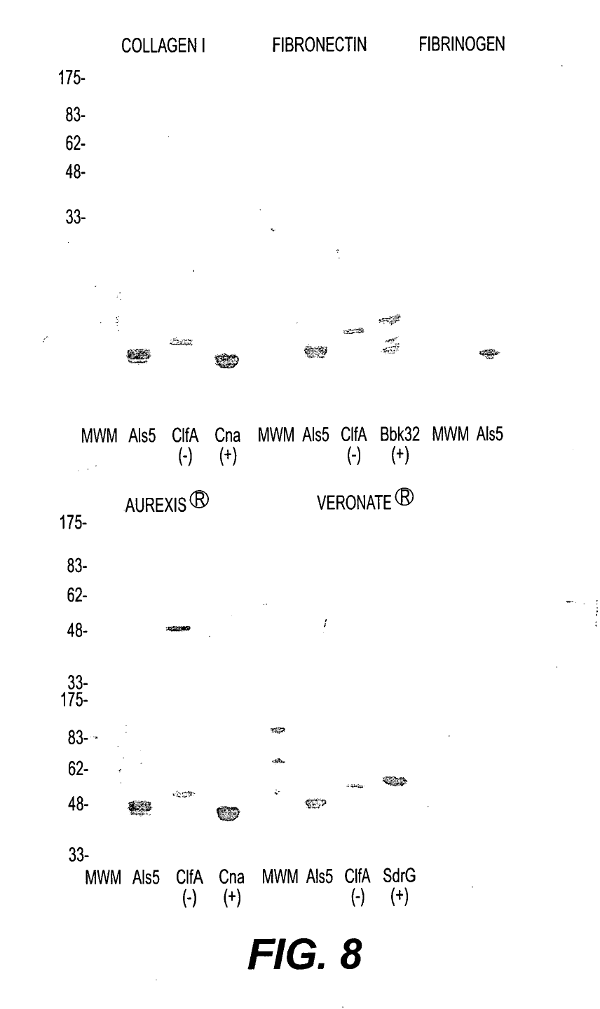

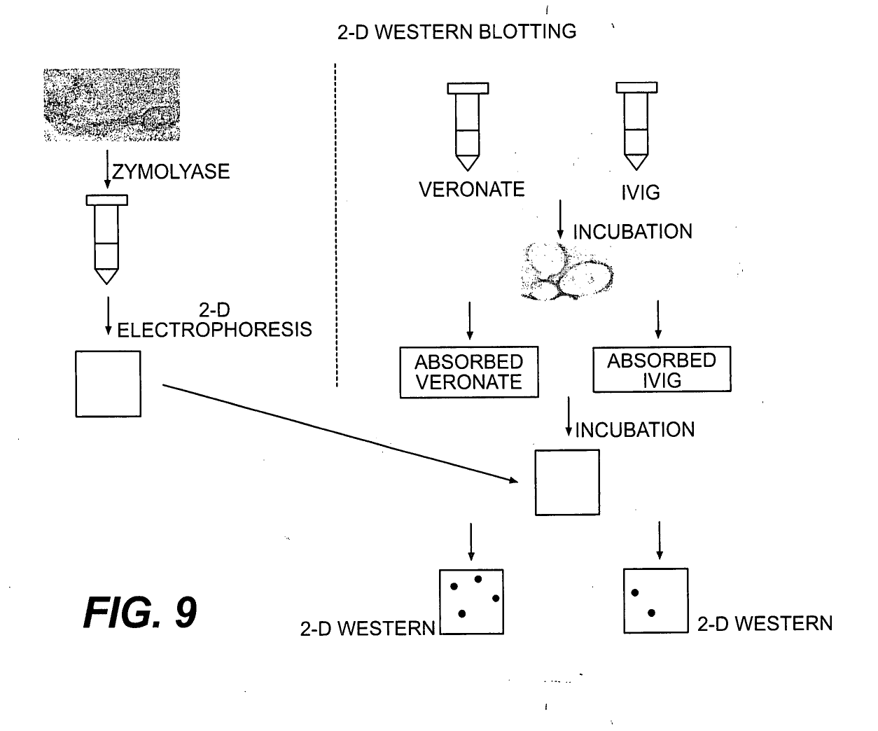

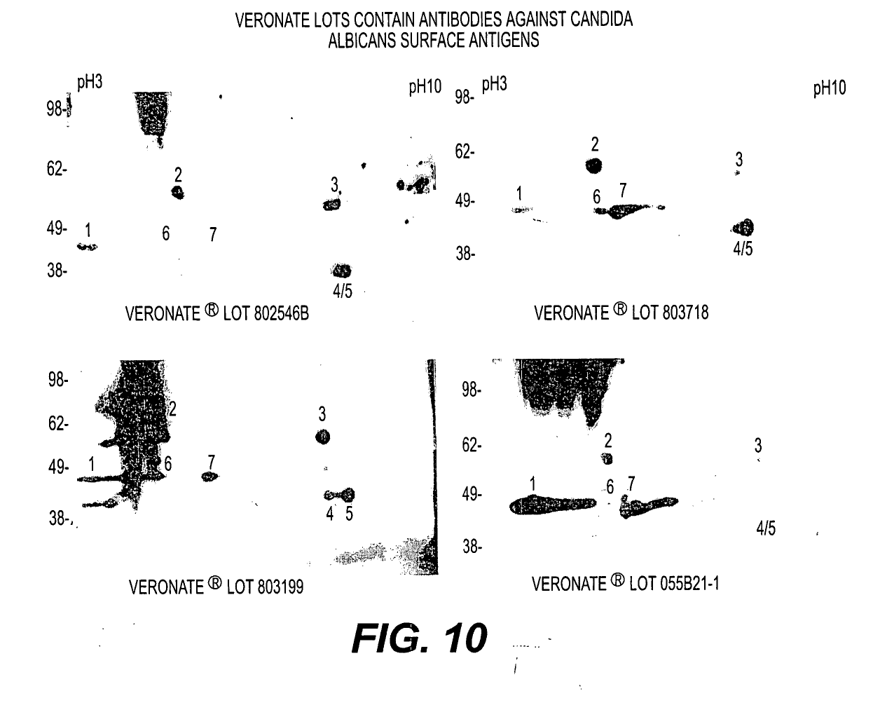

The property of the immunoglobulin compositions of the invention to recognize Candidial surface antigens also makes it possible to identify these surface antigens in accordance with the invention. For example, as shown in FIG. 9, a system of identifying Candida surface antigens is provided wherein a cell wall extract of a Candida yeast such as Candida albicans is separated on a 2-D gel and is then introduced to the immunoglobulin composition in accordance with the invention, in this case, the Veronate® composition which contains higher than normal antibody titers to both ClfA and SdrG. Normal IVIG is used as a control which shows that the Veronate® composition identifies Candidial surface antigens which are not recognized by normal immunoglobulin compositions obtained from pooled immunoglobulin from unselected donors. As shown further below, the immunoglobulin composition of the invention is capable of identifying a high range of surface antigens from Candida yeast. In FIG. 10, for example, immunoblotting tests show that the Veronate® immunoglobulin composition in accordance with the invention contains antibodies which recognize surface antigens from Candida albicans.

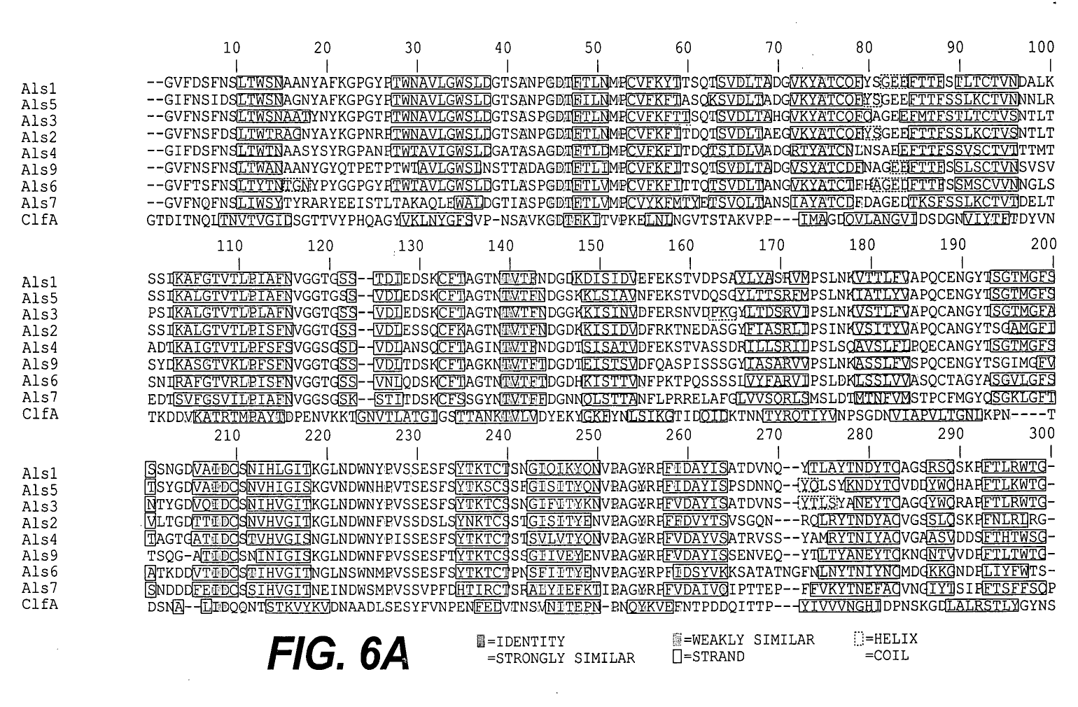

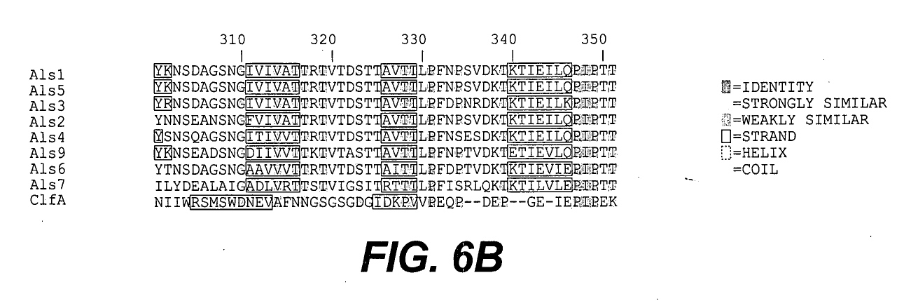

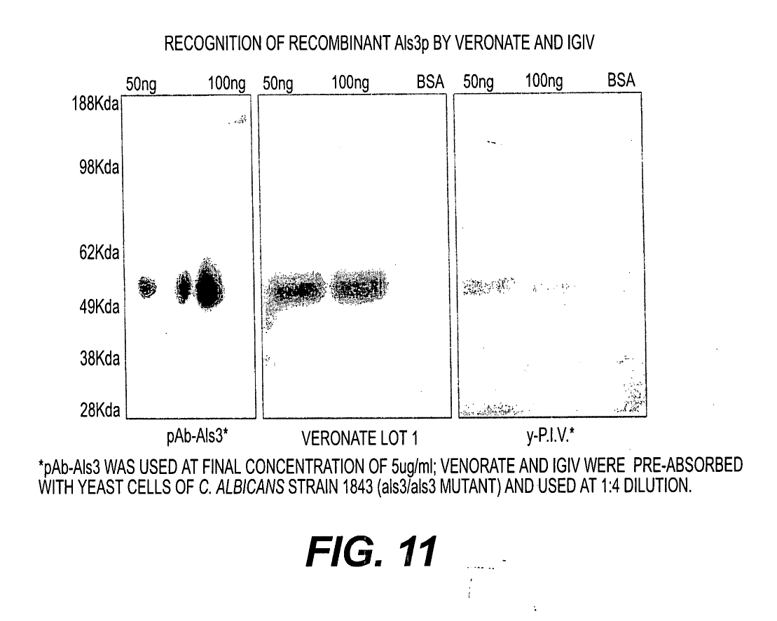

One set of antigens from Candida albicans that is recognized by the immunoglobulin compositions of the present invention is the group of Als proteins whose sequences are shown in FIG. 6. As shown in FIG. 11, Veronate® is capable of recognizing the Als3 protein of Candida albicans, and other tests show that other Als proteins are also recognized by the immunoglobulin compositions in accordance with the invention.

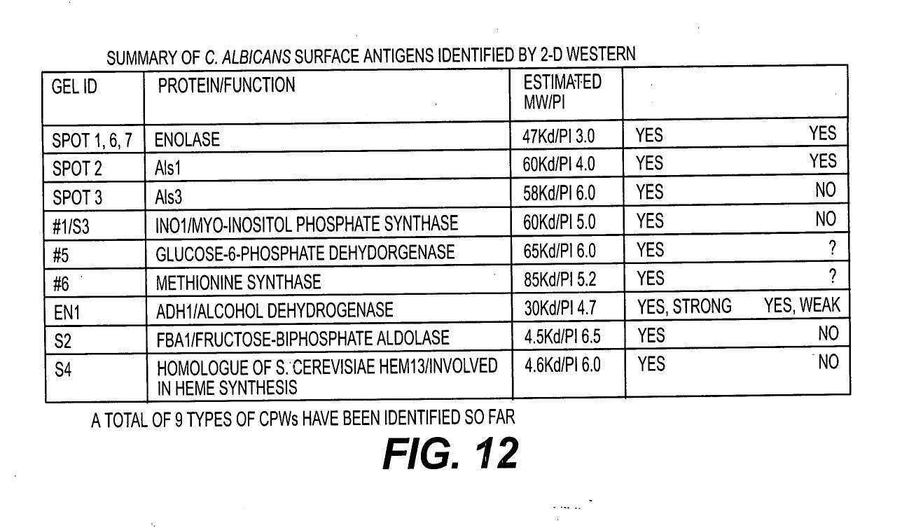

Accordingly, in another embodiment of the present invention, a method of identifying Candidial surface antigens is provided which comprises obtaining a cell wall extract from a culture of Candida yeast cells, introducing into the extract an immunoglobulin composition having an antibody titer to an S. aureus Clumping Factor A (ClfA) protein in combination with an antibody titer to an S. epidermidis serine-aspartate repeat G (SdrG) protein in an amount higher that that found in pooled intravenous immunoglobulin obtained from unselected donors, and detecting Candida antigens that have bound to the antibodies in the immunoglobulin composition. As shown in FIG. 12, Candidial antigens in accordance with the invention which are recognized by the immunoglobulin compositions of the prevent invention include the Als proteins, enolase, INO1-myo-inositol phosphate synthase, glucose-6-phosphate dehydrogenase, methionine synthase, ADH1/alcohol dehydrogenase; FBA1/fructose-biphosphate aldolase and the homologue of S. cerevisiae HEM13 which is involved in HEME synthesis. Probes for identifying Candidial surface antigens are also provided which comprise the immunoglobulin composition as set forth above with a high titer to ClfA and SdrG, and a means for detecting binding of the antibodies in the immunoglobulin composition with Candidial surface antigens.

In still another aspect of the present invention, it has been observed that antibodies (both polyclonal and monoclonal) raised against the MSCRAMM ClfA also are capable of binding to an antigen from Candida albicans, namely an Als protein (see FIG. 8), and thus antibodies to ClfA will also useful in the invention in the identification and inhibition of Als proteins from Candida albicans. Accordingly, in accordance with the present invention, antibodies that recognize both ClfA and Als proteins can be effective in the prevention and treatment of bacterial as well as yeast infections.

In short, the high titer immunoglobulin compositions of the present invention which recognize and which are capable of binding to surface antigens on Candida albicans can be useful in the diagnosis, inhibition, treatment and prevention of infections from Candidial yeast. These compositions will thus also be capable of treating staphylococcal infections at the same time, and their application will be particularly useful in infectious conditions such as late-onset sepsis which affects low and very-low weight newborn infants.

### EXAMPLES

The following examples are provided which exemplify aspects of the preferred embodiments of the present invention and which additional details regarding making and using the invention, and detail the usefulness of the invention in providing methods for treating or preventing Candida species-related infections. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Production of INH-A21 via the Donor Selection Process

Source Plasma

Source Plasma is collected according to the method as disclosed in U.S. Pat. No. 6,692,739, incorporated herein by reference. Source Plasma is obtained from normal, non-immunized donors meeting FDA requirements and iQPP standards for Source Plasma donation. Source Plasma units undergo viral marker testing in accordance with current FDA 21 CFR 640 requirements, that is, the units must be nonreactive or negative for the following:
HBsAg
Anti-HIV-1/2
HIV-1 p24 antigen
Anti-HCV
Syphilis (first donation and every 4 months)

Each plasma unit must also contain an alanine aminotransferase (ALT) level less than twice the upper baseline limit of normal.

Plasma Screening for Antibodies Recognizing MSCRAMM® Proteins ClfA and SdrG

Samples from plasma donors are screened for elevated levels of antibodies to ClfA and SdrG. The screening processes is described in U.S. Pat. No. 6,692,739, incorporated herein be reference. ClfA is described further in U.S. Pat. Nos. 6,008,341 and 6,177,084, incorporated herein by reference. The SdrG protein is described in more detail in U.S. Pat. No. 6,635,473, incorporated herein by reference. Antibody levels specific for ClfA and SdrG in normal IGIV products were previously established by testing commercially available preparations of IGIV and creating a standard reference.

Measurement of Anti-ClfA Titers

Human plasma samples are screened by Inhibitex using an ELISA procedure to assess anti-ClfA (Clf40; amino acids 40-559) MSCRAMM® IgG concentration. Costar plates (VWR# 29442-314) or the equivalent are incubated with a 1 μg/mL Clf40 solution for 12 to 24 hours at 2-8° C. The plates are washed with a solution of PBS and Tween 20, and incubated for 2 to 4 hours at room temperature with a 1% solution of BSA. Following the incubation, plates are washed and the coated wells are overlaid with a 1:200 dilution of each sample to be tested, a Clf40 Calibration Standard (Staphgam MS501, obtained from Cangene Corp., Winnipeg, Canada), or a quality control sample. Plates are incubated for 55 to 65 minutes at room temperature and washed. The plates are incubated for 55 to 65 minutes at room temperature with alkaline phosphatase conjugated goat anti-human IgG (Rockland, Gilbertsville, Pa.) or equivalent, then washed. The plates are subsequently developed for 45 to 55 minutes at room temperature with a 1 mg/mL solution of p-nitrophenylphosphate, disodium (Sigma, St. Louis, Mo.) or the equivalent prepared in diethanolamine substrate buffer (Pierce, Rockford, Ill.). Following plate development, 50 μL of 2N NaOH (LabChem Inc., Pittsburgh, Pa.) is added to each well. Plates are read at 405 nm with a Spectra MAX 250 Bio-Assay plate reader (Molecular Devices Corp., Sunnyvale, Calif.) or equivalent for the presence of anti-ClfA antibodies.

Measurement of Anti-SdrG Titers

Human plasma samples are screened by Inhibitex using an ELISA procedure to assess anti-SdrG (SdrG; amino acids 50-597) MSCRAMM® IgG concentration. Costar plates (VWR# 29442-314) or the equivalent are incubated With a 1 μg/mL SdrG solution for 12 to 24 hours at 2-8° C. The plates are washed with a solution of PBS and Tween 20, and incubated for 2 to 4 hours at room temperature with a 1% solution of BSA. Following the incubation, plates are washed and the coated wells are overlaid with a 1:200 dilution of each sample to be tested, a SdrG Calibration Standard (INH-A21 Inhibitex, Alpharetta, Ga.), or a quality control sample. Plates are incubated for 55 to 65 minutes at room temperature and washed. The plates are incubated for 55 to 65 minutes at room temperature with alkaline phosphatase conjugated goat anti-human IgG (Rockland, Gilbertsville, Pa.).or equivalent then washed. The plates are subsequently developed for 45 to 55 minutes at room temperature with a 1.0 mg/mL solution of p-nitrophenylphosphate disodium (Sigma, St. Louis, Mo.) or equivalent prepared in diethanolamine substrate buffer (Pierce, Rockford, Ill.). Following plate development, 50 μL of 2N NaOH (LabChem Inc., Pittsburgh, Pa.) is added to each well. Plates are read at 405 nm with a Spectra MAX 250 Bio-Assay plate reader (Molecular Devices Corp., Sunnyvale, Calif.) or equivalent for the presence of anti-SdrG antibodies.

The INH-A21 preparation, containing Candida specific antibodies, and prepared as described above was used in both preclinical animal models and a Phase II clinical trial and was shown to be effective in preventing infections caused by Candida species microorganisms, as shown in the following examples:

### Example 2

### INH-A21 Contains Antibodies That Recognize Candida Surface Antigens

Flow Cytometric Analysis of Candida Cell Surface Antigens

Candida Preparation—Overnight cultures were prepared from a few colonies of each Candida strain picked with an inoculum loop off a streak plate that had been prepared from a frozen stock of the strain. The colonies were used to inoculate 10 ml YPD broth cultures and the cultures were grown at 30° C. with 250 rpm rotation. The following day 4 hr cultures were prepared by mixing 1 ml of overnight culture with 9 ml of fresh YPD broth and growing cultures at 30° C. with 250 rpm rotation.

All cultures were stored on ice after growth period. The cultures were washed twice in cold 1× PBS (10 ml per wash). The cultures was adjusted to an OD600 of 1.5 to 2.0 in 1× PBS. 1 ml of the culture at this concentration were retained for blocking.

Blocking—0.1 mg of purified rabbit IgG was mixed with cells in 1× PBS by vortex and incubated for 30 minutes on ice. At 15 minute intervals each strain was vortexed during each incubation.

Antibody Preparation—A 1 mg/ml dilution of INH-A21 in PBSA (2.5% BSA in 1× PBS) was prepared. In addition, a 0.1 mg/ml INH-A21 dilution followed by ten additional 1:2 dilutions were prepared. Solutions were stored on ice.

Primary Ab Incubation—The assay was performed in Bio Rad titertubes for ease in handling. Using a multi-channel pipette, 20 μl of blocked Candida cells were added each tube. 0.5 ml of INH-A21 dilution was then added to the designated tubes. All tubes were vortexed and incubated on ice for 30 minutes. Cells were vortexed at 15 min. intervals. Following the incubation each tube was centrifuged in plate rotor at 3000 RPM for 10 minutes. The supernatant was decanted by hand for each tube. The Candida cells were washed twice in cold PBSA.

Secondary PE F(ab′)₂ Incubation—Each tube received 0.5 ml of a 1:200 dilution of PE conjugated goat F(ab′)₂ anti-human IgG(H+L) secondary antibody. The Candida cells were resuspended and mixed by vortexing. The tubes were incubated on ice for 30 minutes vortexing twice at ten minute intervals. Following this incubation the Candida cells were washed twice with a final resuspension in PBSA. The tubes were stored on ice until FACS analysis.

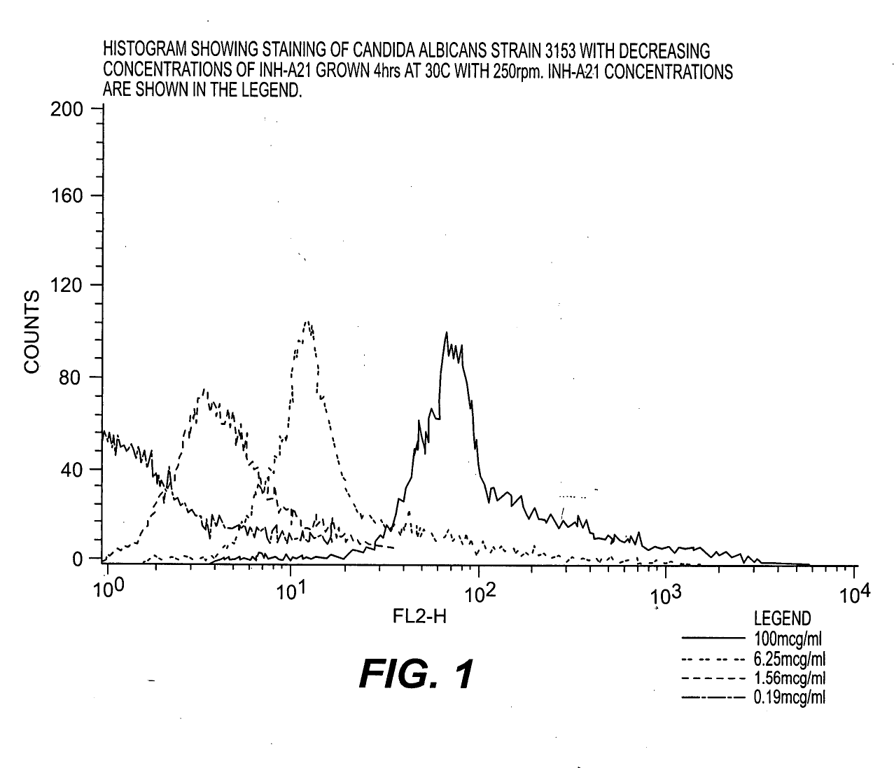

FACS Analysis—Each titertube was transferred to a 12×75 mm flow tube. The FL-2 detector was adjusted so that the isotype control PE emission was detected in the first decade of the FL-2 histogram scale (FIG. 1).

### Example 3

### Antibodies in INH-A21 Specifically Recognize C. albicans Cell Wall Antigens Identification of Immunoreactive Antigens from C. albicans

Cell cultures. Candida albicans s.c 5314 cells were cultured in YPD at 30° C. and used as whole yeast cell to absorb INH-A21 (Lots 802546B & 803718) or normal immunoglobulin(Gammagard S/D, Lot 02129AX11, Baxter Healthcare & Gamma-PIV, Lot X517911, Aventis Behring). The cell pellet from the YPD culture was re-suspended in Lee's medium (Ref) and cultured for 6 hrs at 30° C. Cells from the Lee's medium were re-suspended in sterile water and incubated at 4° C. for 3 days for “starvation”. After the starvation, cells were cultured in Lee's medium at 37° C. for 6 hrs. to induce the formation of hyphae.

Preparation of cell wall extracts. The yeast cells were treated in 20 mM phosphate buffer (pH 7.2) containing 1M sorbitol, 20 mM DTT, complete proteinase inhibitor cocktail, and 10 mg/ml zymolyase 20-T for 1 hr at 37° C. After the treatment, the tube was spun at top speed in a benchtop microcentrifuge for 15 min. and the supernatant was collected.

Absorption of immunoglobulins by the whole yeast cells. About 25 mg of each immunoglobulin sample was mixed with 0.1 ml pellet of the yeast cell and incubated at 4° C. for 6 hrs. The yeast cells were removed by centrifugation at top for 10 min. in a benchtop microcentrifuge and the immunoglobulins were collected.

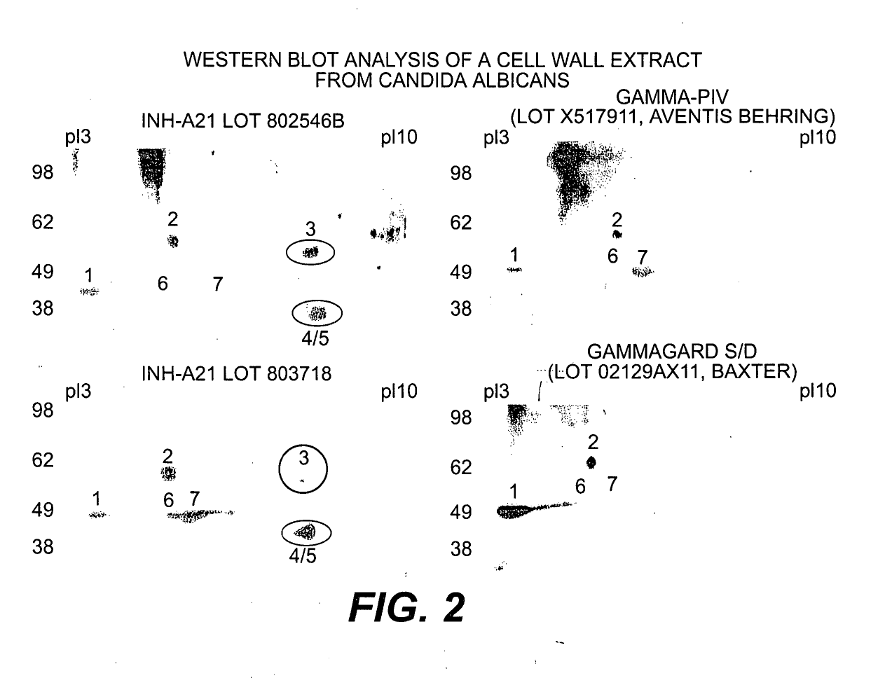

2-D gel electrophoresis and Western blotting. Cell wall extracts from the hyphae of Candida albicans s.c. 5314 were separated by 2-D gel electrophoresis in the ZOOM IPGRunner System (Invitrogen) according to the instruction. The proteins were transferred onto PVDF membrane and incubated with the yeast-absorbed immunoglobulins. The mouse anti-human IgG conjugated to HRP was used as secondary antibody and the protein spots were detected with the Supersignal pico west substrate (PIRECE) (FIG. 2.) These data show that INH-A21 prepared in accordance with the invention contains antibodies to Candida antigens, which are not present in the unselected, normal commercially available immune globulin products. The antigens (number 3, 4, and 5) isolated at 50-65 Kd, pl 7-9 and at 40-49 kd and pl 7-9 specifically reacted with INH-A21 and not normal immune globulins.

### Example 4

### Identification of Immunoreactive Candida Antigens

To further identify the antigens that are recognized preferentially by INH-A21, cell wall extracts from the Candida albicans s.c. 5314 were separated by 2-D gel electrophoresis in the ZOOM IPGRunner System (Invitrogen) according to the instruction. Duplicate 2D gels were run using the cell wall extracts. One gel was transferred to nitrocellulose and probed as described in Example 3 then visualized on Kodak BioMax XAR film. The second gel was stained with colloidal Coomassie Blue to visualize the bands. In order to excise the band corresponding to individual Western spots, a second film was produced. The immunoreactive bands of interest were cut out of this film and overlaid onto the stained gel. Using the hollowed out film as a guide, a sterile, disposable scalpel blade was used to excise the stained band of interest. The gel slice was placed in a clear, dry microfuge tube that was previously rinsed with HPLC grade water and HPLC grade methanol. The gel slice was than washed twice for 2 minutes each with 0.5 mL of 50% acetonitrile in HPLC grade water. After removing the excess liquid, the moist gel slice was stored at −80° C. until analyzed. Additionally, an equivalent unstained area of the gel was excised and stored in the same manner to control for chemical and generalized nonspecific protein background noise during analysis. The excised band was homogenized in an aqueous buffer and subjected to proteolysis using trypsin. The proteolyzed sample was then filtered and analyzed by microcapillary reverse-phase HPLC nano-spray tandem mass spectrometry on a Finnigan LCQ DECA XP Plus quadrupole ion trap mass spectrometer. This instrument configuration is capable of acquiring individual sequence (MS/MS) spectra on-line at high sensitivity (<<1 femtomole) for multiple peptides in the chromatographic run. These MS/MS spectra are correlated with known sequences using the algorithm Sequest developed at the University of Washington (16), and by programs developed at Harvard (17). The MS/MS peptide sequences were reviewed by a scientist for consensus with known proteins and the results manually confirmed for fidelity. The proteins identified from the 2D gel were Als1 (spot 2), Als3 (spot 3), and enolase (spot 1).

More information concerning the Als proteins and antibodies thereto is disclosed in U.S. patent application Pub. No. 2003/0124134, said application incorporated herein by reference.

### Example 5

### Activity of INH-A21 in an Experimental Model of Prophylaxis Against Candida Systemic Infection

Mice were used in preclinical studies of INH-A21 to explore the prophylactic activity of INH-A21 an in vivo model of Candida mediated mortality. To demonstrate biologic activity conferred by the selection of antibodies against SdrG and ClfA, with cross-reactivity against Candida antigens, Candida albicans was used as the infecting organism in this model.

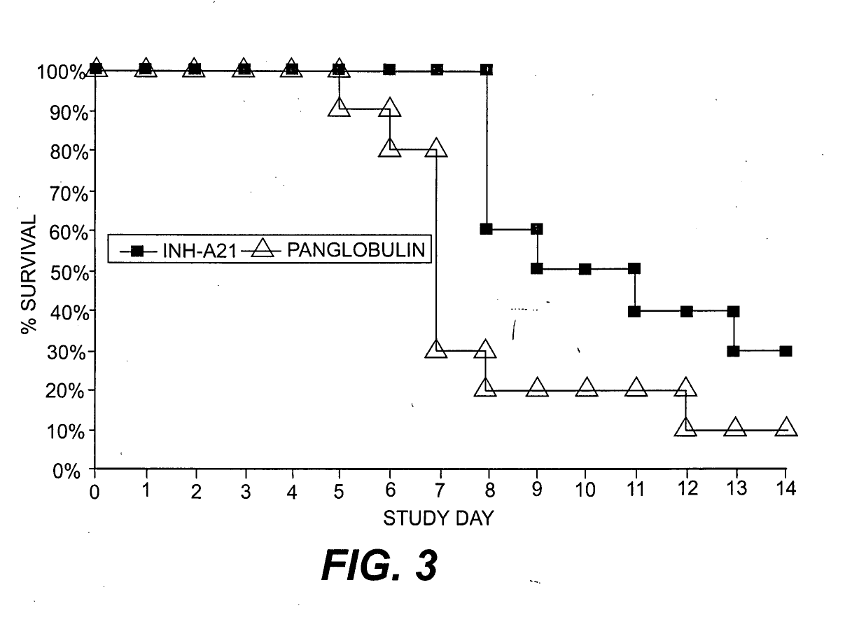

Mice were administered INH-A21 or a non-donor selected or normal IGIV (Panglobulin®, American Red Cross Plasma Services). INH-A21 or Panglobulin® was given as a single dose of 12.5 mg/kg. Eighteen hours after dosing, animals were challenged intravenously with Candida albicans. Efficacy was measured as survival at 14 days following the fungal challenge. These data demonstrate that INH-A21 protects against IV challenge with C. albicans (FIG. 3).

### Example 6

### Clinical Trials in Premature Infants with Immune Globulins

With the exception of elevated levels of antibodies to ClfA, SdrG, and Candidial antigens, INH-A21 is a human IGIV manufactured by methods common to other commercially available IGIV products. A review of the efficacy of prior clinical trials in premature infants using these products is appropriate to evaluate the potential efficacy in this population. In a study reported by Baker et al., 287 infants (birth weight 500 to 1750 grams) received 1,125 infusion of IGIV, and 297 infants received 1,163 infusions of placebo.¹³ There was not a reduction in the incidence of late-onset fungal sepsis between the IVIG group and placebo. Weisman et al. randomized 372 infants (birth weight 560 to 2000 grams) to receive IGIV and 381 to receive albumin.¹⁴ There was not a reduction in the incidence of late-onset fungal sepsis between the IVIG group and placebo. In a study by Fanaroff et al., 1204 infants received IGIV (n=1204), while control infants (n=1212) received albumin, or during a second phase of the study, no infusion.¹⁵ There was not a reduction in the incidence of late-onset fungal sepsis between the IVIG group and placebo.

INH-A21 Reduces Candida Infection in VLBW Infants.

In a Phase II, placebo-controlled, double-blind study in VLBWI (500-1,250 g), prophylactic administration of INH-A21 (750 mg/kg) was shown to reduce the incidence of late-onset candidial sepsis by 67% when compared to placebo (Table 1). In addition to reducing the incidence of candidemia, INH-A21 also reduced the incidence of S. aureus infection by 63%, and all-cause mortality by 36%. Despite the numerous clinical trials conducted with immune globulins in neonates, the magnitude in the reduction of candidial infection demonstrated with INH-A21 in this clinical trial has not been seen previously.

TABLE 1
Percent of Infants with Infections or Mortality

| | | INH-A21 | Percent |
|---|---|---|---|
| | Placebo | (750 mg/kg) | Reduction |
| Number of Infants | 158 | 157 | |
| Candida sp. | 5.7% | 1.9% | 67% |
| S. aureus | 7.0% | 2.5% | 63% |
| Mortality | 7.0% | 4.0% | 36% |

### Example 7

### Studies of Als Proteins from the Surface of Candida albicans

Antibodies generated against a previously discovered family of structurally related surface proteins (MSCRAMMs) on Gram-positive bacteria have been shown to be useful as preventive and therapeutic agents (Hall et al., 2003. Infect. Immun. 71, 6864-6870). We have recently recognized that a previously described family of proteins on the surface of Candida albicans, the Als proteins, is structurally and immunologically similar to the bacterial MSCRAMMs. Like MSCRAMMs, the Als cell-surface proteins have a multi-domain structure. Each Als protein has a very well conserved N-terminal domain followed by a central domain formed by tandemly repeated motifs and a serine-threonine-rich C-terminal domain that can be variable in length. The C-terminal residues of the mature Als proteins are covalently linked to the C. albicans cell wall by β1, 6-glucan chains. Similarity of the Als N-terminal domains to the sequence of Saccharomyces cerevisiae α-agglutinin and Staphylococcus aureus ClfA, both cell-surface adhesins, suggests that the Als proteins are adhesins, mediating the C. albicans attachment to host surfaces.

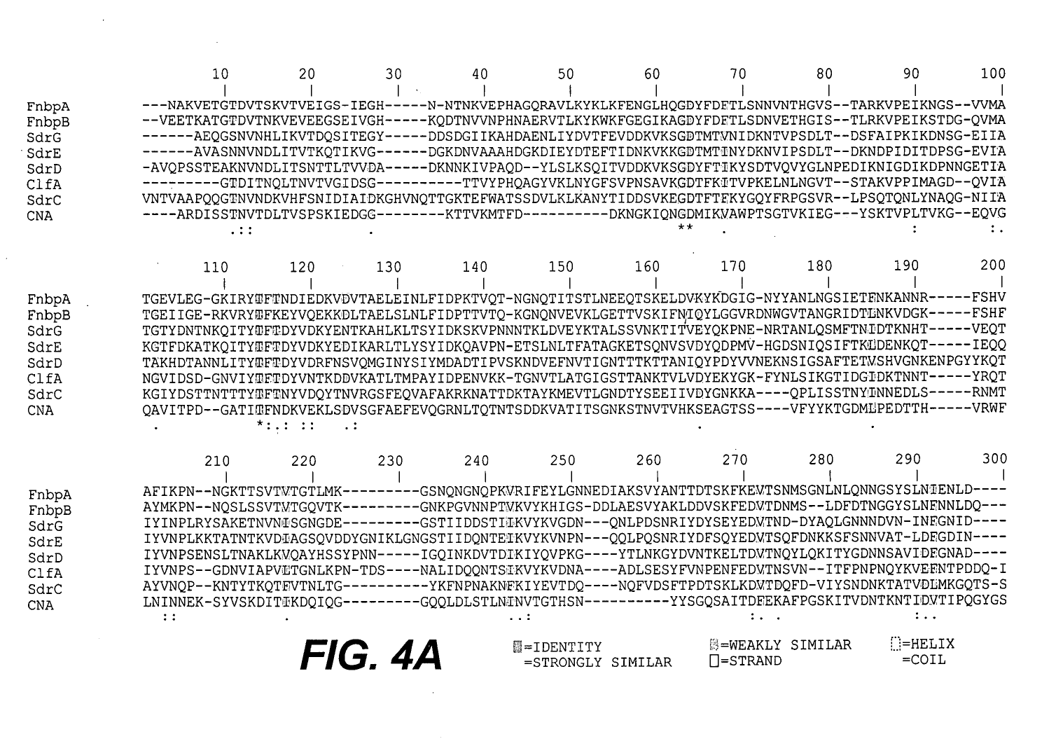

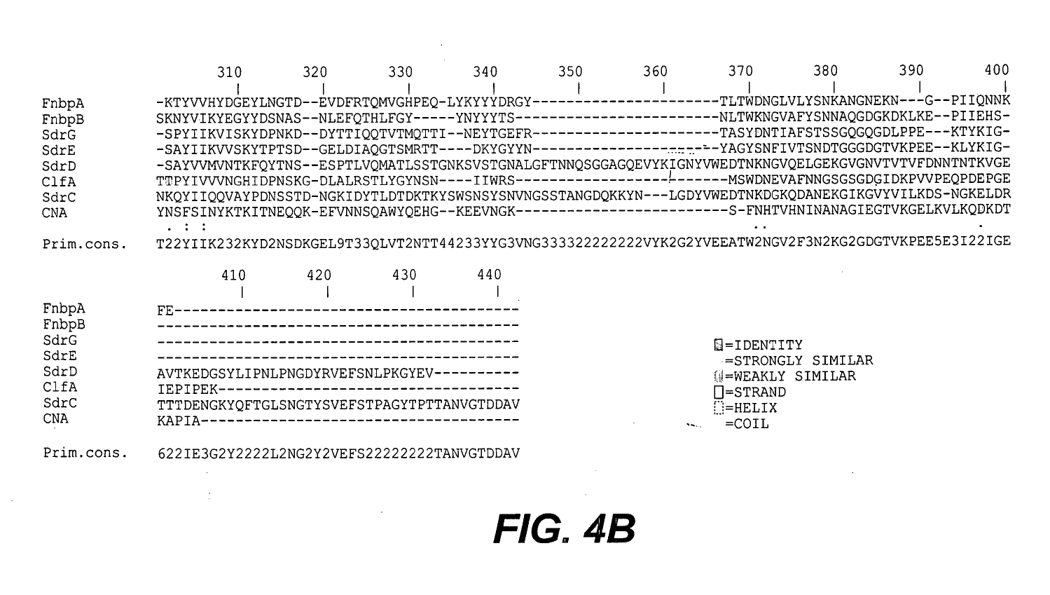

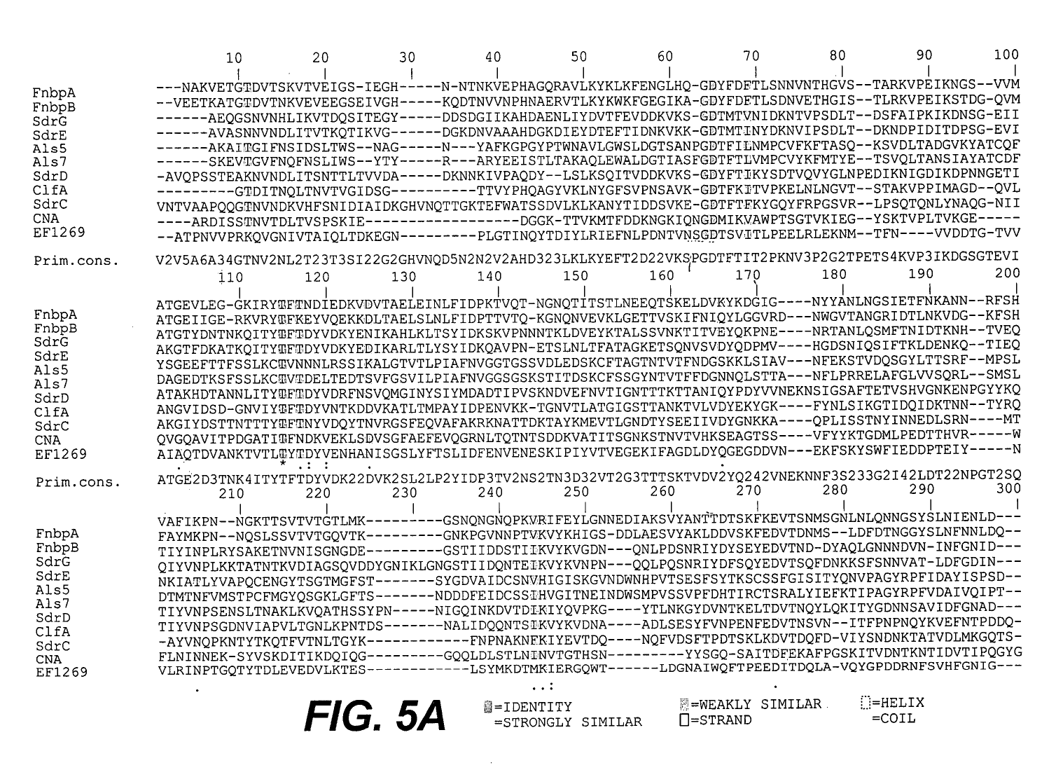

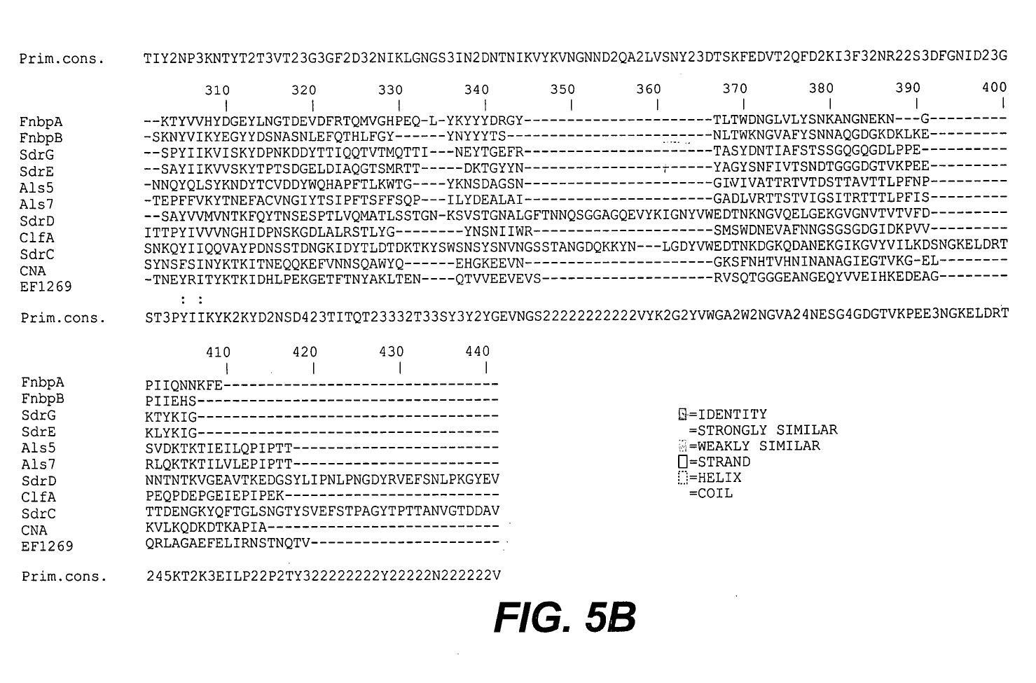

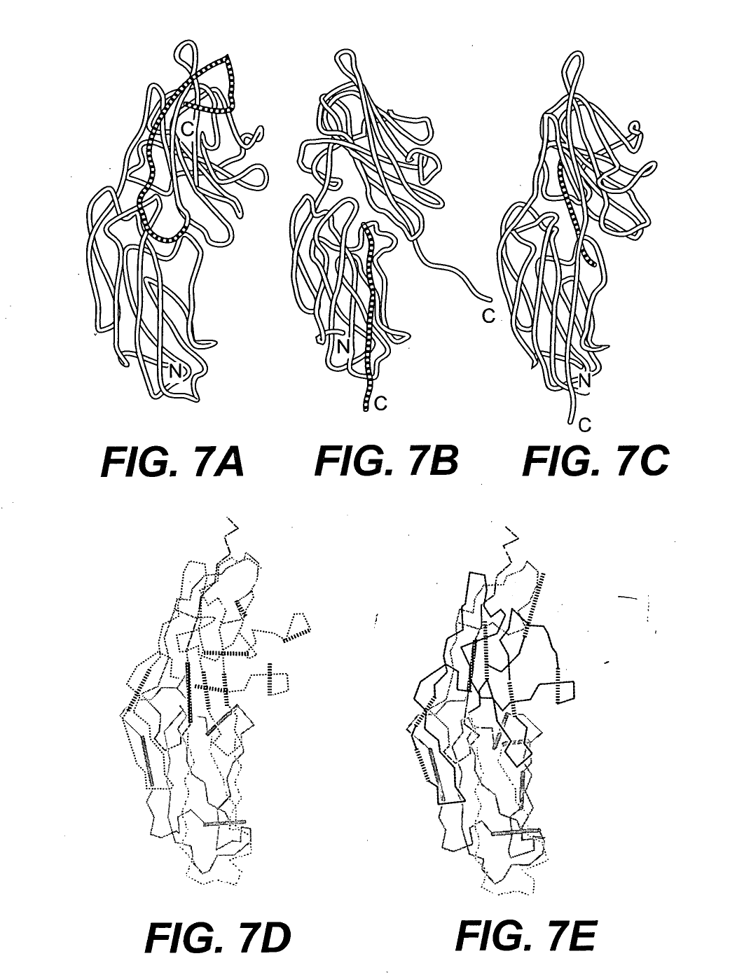

The use of polyclonal or monoclonal antibodies reacting with Als proteins constitutes a new potential strategy for the prevention and treatment of infections caused by C. albicans and related organisms. An analogous strategy, using antibodies targeted to the MSCRAMM ClfA, has been effective in animal models for the treatment and prevention of infections caused by S. aureus. The overall structural features of the Als proteins are strikingly similar to those found in bacterial MSCRAMMs. As shown on FIG. 4, the amino acid sequences of the bacterial ligand-binding regions are well conserved. Interestingly, the amino acid sequence of the proposed ligand-binding N-terminal domain of the Als proteins is 15-20% identical to the corresponding region of ClfA. FIG. 5 shows the amino acid similarity between the N-terminal region of Als5 and Als7 and the bacterial proteins. This homology extends to the folding prediction of these C. albicans proteins, secondary and tertiary structural predictions suggest that the N-terminal region of the Als proteins fold into tandem IgG-like domains, a structural fold that is present in the described bacterial MSCRAMMs (FIGS. 6 and 7). In fact, the conserved sequence motif GDTF (amino acids 65-68 in Als5) and a variation of the ClfA IYTFTDYVN motif (amino acids 112-122 in Als5) are present in the Als proteins and in the bacterial MSCRAMMs. These sequences define the latching trench critical in the “dock, lock and latch” model of ligand binding used by the MSCRAMM and constitute structural features that suggest a common ligand binding mechanism between MSCRAMMs and Als proteins (Ponnuraj et. al., 2003 Cell 115, 217-228). In summary, the observed similarity between the bacterial and yeast proteins show that the IgG-like domains of C. albicans cell-surface Als proteins can interact with ligand peptides and that antibodies generated against these recombinant domains will be useful in blocking ligand binding and as preventive or therapeutic agents.

One of the most surprising and unexpected observations that was made is that antibodies (both polyclonal and monoclonal) raised against an MSCRAMM (ClfA in this example) can recognize a recombinant version of an Als protein (FIG. 8). Although the putative ligand binding domains of MSCRAMMs and Als proteins have amino acid sequences that are 15-20% identical and the projected structural folds are similar, the immunological cross-reactivity is remarkable and unexpected. This observation indicates that mAbs such as the mAb which recognizes ClfA can also recognize antigens from C. albicans such as the Als proteins and thus can be effective in the prevention and treatment of bacterial as well as yeast infections.

We have cloned the regions of the Als5 and Als7 genes predicted to encode the N-terminal regions homologous to the S. aureus ClfA ligand-binding domain. These constructs encompass amino acids 22-371 in Als5 and 23-372 in Als7. Initially, these constructs were expressed in E. coli, however, to promote native protein folding, they will be overexpressed in a yeast heterologous host, Pichia pastoris.

In addition, we have performed experiments showing that the recombinant Als5 N-terminal domain binds to extracellular matrix proteins such as fibrinogen, fibronectin and collagen Type I (FIG. 8A). Furthermore, because of the topological similarity between Als5 and ClfA, we tested the ability of antibodies generated against ClfA to recognize Als5. The results are shown in FIG. 8, demonstrating that the antibodies to ClfA recognize Als proteins including Als5, and that the commercial preparation of polyclonal and monoclonal antibodies designed to target the S. aureus MSCRAMM ClfA crossreact with Als5 and can thus be used to inhibit both S. aureus and C. albicans.

Other aspects of the present experiments include the overexpression of Als5 and Als7 (the most and least conserved sequences in the Als family), and the generation of both monoclonal and polyclonal antibodies against these recombinant proteins. In accordance with the invention, antibodies to Als proteins such as Als5 and Als7 will be useful in the inhibition of C. albicans and the treatment and prevention of C. albicans infections.

The present invention is thus particularly useful because of the fact that since infections caused by C. albicans and related organisms are difficult to diagnose, these organisms can survive in the host without causing detectable disease symptoms, or can cause symptoms that vary in site and severity. C. albicans has numerous mechanisms to adapt in the host, including differential gene expression that leads to switching between two morphologies: the blastospore (yeast form) and filamentous forms (hyphae and pseudohyphae). The ability to change from a blastospore to a filamentous form is a key virulence factor, since it has been shown that the hyphae and pseudohyphae are the virulent, invasive forms that cause disease (Hoyer L. L. et al., 1995. Mol. Microbiol 15: 39-54). The identification and characterization of hyphae-surface proteins involved in virulence, such as the Als proteins, enables immunotherapeutic strategies that are superior to existing antifungal agents. While existing antifungal agents are microbicidal for Candida in vitro, the attributable mortality for candidemia is 38%, even during treatment with antifungal agents such as amphotericin B. Therefore, either passive or active immunotherapy to treat or prevent disseminated candidiasis in accordance with the invention will be a promising complement or alternative to standard antifungal therapy.

The existing patent US2003/0124134 A1, incorporated herein by reference, describes pharmaceutical compositions and methods to vaccinate against candidiasis using Als1 N-terminal region (amino acids 17-432) as an immunogen. However, in accordance with the present invention, the described constructs for Als5(22-371) and Als7(23-372) may be used as immunogens, and this will likely give a more effective preparation because:

(a) they represent a structurally defined region homologous to those present in bacterial proteins of similar function (FIG. 5),

(b) they bind to extracellular matrix proteins (FIG. 8A) that may mediate C. albicans attachment to host tissues and protein-coated biomaterials, and

(c) antibodies present in Veronate® and Aurexis® recognize them (FIG. 8B).

As referred to in this application, Veronate® refers to a donor selected or donor-stimulation human immunoglobulin composition such as described in U.S. Pat. No. 6,692,739, incorporated herein by reference, and Aurexis® refers to a composition comprising a monoclonal antibody to ClfA as disclosed in co-pending U.S. patent Ser. No. 10/156,052, published as U.S. patent application Pub. 2003/0099656, incorporated herein by reference.

The advantage of using the Als5 and Als7 N-terminal regions and antibodies generated against them as a treatment strategy for the prevention of C. albicans infections is that the humanized antibodies are very effective and do not cause secondary adverse reactions. This is a significant improvement over the antifungal therapies that can be toxic to the host at high or prolonged doses.

### REFERENCES

The following references referred to above are hereby incorporated into the above specification as if set forth in their entirety:
1. Siber G R, Leszcynski J, Pena-Cruz V, et al. Protective activity of a human respiratory syncytial virus immune globulin prepared from donors screened by microneutralization assay. J Infect Dis 1992; 165:456-63.
2. Reduction of respiratory syncytial virus hospitalization among premature infants and infants with bronchopulmonary dysplasia using respiratory syncytial virus immune globulin prophylaxis. The PREVENT Study Group. Pediatrics 1997; 99:93-9.
3. Groothuis J R, Gutierrez K M, Lauer B A. Respiratory syncytial virus infection in children with bronchopulmonary dysplasia. Pediatrics 1988; 82:199-203.
4. Green M, Brayer A F, Schenkman K A, Wald E R. Duration of hospitalization in previously well infants with respiratory syncytial virus infection. Pediatr Infect Dis J 1989; 8:601-5.
5. McIver J, Grady G. Immunoglobulin Preparations. In: Churchill W H, Kurtz S R, eds. Transfusion Medicine. Boston: Blackwell, 1988.
6. Schneider L, Geha R. Outbreak of Hepatitis C Associated with Intravenous Immunoglobulin Administration—United States October 1993-June 1994. MMWR Morb Mortal Wkly Rep 1994; 43:505-509.
7. Edwards C A, Piet M P J, Chin S, al. e. Tri(n Butyl)Phosphate Detergent Treatment of Licensed Therapeutic and Experimental Blood Derivatives. Vox Sang 1987; 52:53-59.
8. Groothuis J R, Simoes E A, Lehr M V, et al. Safety and bioequivalency of three formulations of respiratory syncytial virus-enriched immunoglobulin. Antimicrob Agents Chemother 1995; 39:668-71.
9. Groothuis J R, Simoes E A, Levin M J, et al. Prophylactic administration of respiratory syncytial virus immune globulin to high-risk infants and young children. The Respiratory Syncytial Virus Immune Globulin Study Group. N Engl J Med 1993; 329:1524-30.
10. Ellenberg S S, Epstein J S, Fratantoni J C, Scott D, Zoon K C. A trial of RSV immune globulin in infants and young children: the FDA's view. N Engl J Med 1994; 331:203-5.
11. CDC. Staphylococcus aureus resistant to vancomycin—United States, 2002. MMWR Morb Mortal Wkly Rep 2002; 51:565-7.
12. Garrett D O, Jochimsen E, Murfitt K, et al. The emergence of decreased susceptibility to vancomycin in Staphylococcus epidermidis. Infect Control Hosp Epidemiol 1999; 20:167-70.
13. Baker C J, Melish M E, Hall R T, Casto D T, Vasan U, Givner L B. Intravenous immune globulin for the prevention of nosocomial infection in low-birth-weight neonates. The Multicenter Group for the Study of Immune Globulin in Neonates. N Engl J Med 1992; 327:213-9.
14. Weisman L E, Stoll B J, Kueser T J, et al. Intravenous immune globulin prophylaxis of late-onset sepsis in premature neonates. J Pediatr 1994; 125:922-30.
15. Fanaroff A A, Korones S B, Wright L L, et al. A controlled trial of intravenous immune globulin to reduce nosocomial infections in very-low-birth-weight infants. National Institute of Child Health and Human Development Neonatal Research Network. N Engl J Med 1994; 330:1107-13.
16. Eng J K, McCormick A L and Yates J R III. An approach to correlate tandem mass spectral data of peptides with amino acid sequences in a protein database. (1994) J. Am. Soc. Mass Spectrom. 5: 976-989.
17. Chittum H S, Lane W S, Carlson B A, Roller P P, Lung F D, Lee B J, Hatfield D L. Rabbit β-globin is extended beyond its UGA stop codon by multiple suppressions and translational reading gaps. (1998) Biochemistry 37:10866-10870.

1. A method of inhibiting a Candidial infection comprising administering to a patient in need thereof a purified human donor immunoglobulin composition having an antibody titer to an S. aureus Clumping Factor A (ClfA) protein in combination with an antibody titer to an S. epidermidis serine-aspartate repeat G (SdrG) protein wherein both antibody titers are higher than that found in pooled intravenous immunoglobulin obtained from unselected human donors in an amount effective to inhibit a Candidial infection.

2. The method of claim 1 wherein the human donor immunoglobulin composition is obtained by selecting donor having an antibody titer to ClfA and SdrG in an amount that is higher than that found in pooled intravenous immunoglobulin obtained from unselected human donors.

3. The method of claim 1 wherein the human donor immunoglobulin composition is obtained by a method comprising administering ClfA and SdrG to a human host donor in an amount sufficient to induce an antibody titer to these adhesins that is higher that found in pooled intravenous immunoglobulin obtained from unselected donors, obtaining blood or plasma from said donors, and purifying said blood or plasma to obtain a purified human donor immunoglobulin having antibody titers to ClfA and SdrG in an amount which is higher than that found in pooled intravenous immunoglobulin obtained from unselected human donors.

4. The method of claim 1 wherein the human donor immunoglobulin composition has an antibody titer to ClfA and SdrG which is at least twice than that found in pooled intravenous immunoglobulin from unselected human donors.

5. The method of claim 1 wherein the human donor immunoglobulin composition also has an antibody titer to a Candida albicans surface antigen in an amount that is higher than that found in pooled intravenous immunoglobulin obtained from unselected human donors.

6. The method of claim 1 wherein the Candidial infection is caused by Candida albicans.

7. A method of diagnosing a Candidial infection comprising introducing an immunoglobulin composition having an antibody titer to an S. aureus Clumping Factor A (ClfA) protein in combination with an antibody titer to an S. epidermidis serine-aspartate repeat G (SdrG) protein in an amount higher that that found in pooled intravenous immunoglobulin obtained from unselected donors to a sample suspected of containing antigens from Candida yeast for a time sufficient to allow the Candida antigens to bind to the antibodies in the immunoglobulin composition, and diagnosing a Candidial infection by determining if Candida antigens in the sample have bound to the antibodies in the immunoglobulin composition.

8. The method of claim 7 wherein the Candidial infection is caused by Candida albicans.

9. A method of identifying a Candidial surface antigen comprising obtaining a cell wall extract from a culture of Candida yeast cells, introducing into the extract an immunoglobulin composition having an antibody titer to an S. aureus Clumping Factor A (ClfA) protein in combination with an antibody titer to an S. epidermidis serine-aspartate repeat G (SdrG) protein in an amount higher that that found in pooled intravenous immunoglobulin obtained from unselected donors, and detecting Candida antigens that have bound to the antibodies in the immunoglobulin composition.

10. The method of claim 9 wherein the Candidial surface antigen is selected from the group consisting of Als proteins, enolase, INO1-myo-inositol phosphate synthase, glucose-6-phosphate dehydrognease, methionine synthase, ADH1/alcohol dehydrogenase; FBA1/fructose-biphosphate aldolase and the homologue of S. cerevisiae HEM13 which is involved in HEME synthesis.

11. A probe for identifying Candidial surface antigens comprising an immunoglobulin composition having an antibody titer to an S. aureus Clumping Factor A (ClfA) protein in combination with an antibody titer to an S. epidermidis serine-aspartate repeat G (SdrG) protein in an amount higher that that found in pooled intravenous immunoglobulin obtained from unselected donors and a means for detecting binding of the antibodies in the immunoglobulin composition with Candidial surface antigens.

12. A method of inhibiting an infection caused by Candida albicans comprising administering to a patient in need thereof a purified human donor immunoglobulin composition having an antibody titer to an S. aureus Clumping Factor A (ClfA) protein in combination with an antibody titer to an S. epidermidis serine-aspartate repeat G (SdrG) protein wherein both antibody titers are higher than that found in pooled intravenous immunoglobulin obtained from unselected human donors in an amount effective to inhibit an infection caused by Candida albicans.

13. The method of claim 12 wherein said method is used to treat or prevent an infection caused by Candida albicans.

14. A method of inhibiting an infection caused by Candida albicans comprising administering to a patient in need thereof an effective amount of a purified human donor immunoglobulin composition having an antibody titer to a Candida albicans surface antigen in an amount that is higher than that found in pooled intravenous immunoglobulin obtained from unselected human donors.

15. The method of claim 14 wherein the surface antigen is selected from the group consisting of Als proteins, enolase, INO1-myo-inositol phosphate synthase, glucose-6-phosphate dehydrognease, methionine synthase, ADH1/alcohol dehydrogenase; FBA1/fructose-biphosphate aldolase and the homologue of S. cerevisiae HEM13 which is involved in HEME synthesis.

16. A method of inhibiting a Candidial infection comprising administering to a patient in need thereof a purified human donor immunoglobulin composition having an antibody titer to an S. aureus Clumping Factor A (ClfA) protein in an amount that is higher than that found in pooled intravenous immunoglobulin obtained from unselected human donors in an amount effective to inhibit a Candidial infection.


SEQUENCE LISTING (19 sequences)

SEQ ID NO: 1 - protein, 334 aa, Candida albicans
NAKVETGTDVTSKVTVEIGSIEGHNNTNKVEPHAGQRAVLKYKLKFENGLHQGDYFDFTLSNNVNTHGVSTARKVPEIKNGSVVMATGEVLEGGKIRYTFTNDIEDKVDVTAELEINLFIDPKTVQTNGNQTITSTLNEEQTSKELDVKYKDGIGNYYANLNGSIETFNKANNRFSHVAFIKPNNGKTTSVTVTGTLMKGSNQNGNQPKVRIFEYLGNNEDIAKSVYANTTDTSKFKEVTSNMSGNLNLQNNGSYSLNIENLDKTYVVHYDGEYLNGTDEVDFRTQMVGHPEQLYKYYYDRGYTLTWDNGLVLYSNKANGNEKNGPIIQNNKFE

SEQ ID NO: 2 - protein, 332 aa, Candida albicans
VEETKATGTDVTNKVEVEEGSEIVGHKQDTNVVNPHNAERVTLKYKWKFGEGIKAGDYFDFTLSDNVETHGISTLRKVPEIKSTDGQVMATGEIIGERKVRYTFKEYVQEKKDLTAELSLNLFIDPTTVTQKGNQNVEVKLGETTVSKIFNIQYLGGVRDNWGVTANGRIDTLNKVDGKFSHFAYMKPNNQSLSSVTVTGQVTKGNKPGVNNPTVKVYKHIGSDDLAESVYAKLDDVSKFEDVTDNMSLDFDTNGGYSLNFNNLDQSKNYVIKYEGYYDSNASNLEFQTHLFGYYNYYYTSNLTWKNGVAFYSNNAQGDGKDKLKEPIIEHS

SEQ ID NO: 3 - protein, 331 aa, Candida albicans
AEQGSNVNHLIKVTDQSITEGYDDSDGIIKAHDAENLIYDVTFEVDDKVKSGDTMTVNIDKNTVPSDLTDSFAIPKIKDNSGEIIATGTYDNTNKQITYTFTDYVDKYENIKAHLKLTSYIDKSKVPNNNTKLDVEYKTALSSVNKTITVEYQKPNENRTANLQSMFTNIDTKNHTVEQTIYINPLRYSAKETNVNISGNGDEGSTIIDDSTIIKVYKVGDNQNLPDSNRIYDYSEYEDVTNDDYAQLGNNNDVNINFGNIDSPYIIKVISKYDPNKDDYTTIQQTVTMQTTINEYTGEFRTASYDNTIAFSTSSGQGQGDLPPEKTYKIG

SEQ ID NO: 4 - protein, 338 aa, Candida albicans
AVASNNVNDLITVTKQTIKVGDGKDNVAAAHDGKDIEYDTEFTIDNKVKKGDTMTINYDKNVIPSDLTDKNDPIDITDPSGEVIAKGTFDKATKQITYTFTDYVDKYEDIKARLTLYSYIDKQAVPNETSLNLTFATAGKETSQNVSVDYQDPMVHGDSNIQSIFTKLDENKQTIEQQIYVNPLKKTATNTKVDIAGSQVDDYGNIKLGNGSTIIDQNTEIKVYKVNPNQQLPQSNRIYDFSQYEDVTSQFDNKKSFSNNVATLDFGDINSAYIIKVVSKYTPTSDGELDIAQGTSMRTTDKYGYYNYAGYSNFIVTSNDTGGGDGTVKPEEKLYKIG

SEQ ID NO: 5 - protein, 408 aa, Candida albicans
AVQPSSTEAKNVNDLITSNTTLTVVDADKNNKIVPAQDYLSLKSQITVDDKVKSGDYFTIKYSDTVQVYGLNPEDIKNIGDIKDPNNGETIATAKHDTANNLITYTFTDYVDRFNSVQMGINYSIYMDADTIPVSKNDVEFNVTIGNTTTKTTANIQYPDYVVNEKNSIGSAFTETVSHVGNKENPGYYKQTIYVNPSENSLTNAKLKVQAYHSSYPNNIGQINKDVTDIKIYQVPKGYTLNKGYDVNTKELTDVTNQYLQKITYGDNNSAVIDFGNADSAYVVMVNTKFQYTNSESPTLVQMATLSSTGNKSVSTGNALGFTNNQSGGAGQEVYKIGNYVWEDTNKNGVQELGEKGVGNVTVTVFDNNTNTKVGEAVTKEDGSYLIPNLPNGDYRVEFSNLPKGYEV

SEQ ID NO: 6 - protein, 332 aa, Candida albicans
GTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPEK

SEQ ID NO: 7 - protein, 407 aa, Candida albicans
VNTVAAPQQGTNVNDKVHFSNIDIAIDKGHVNQTTGKTEFWATSSDVLKLKANYTIDDSVKEGDTFTFKYGQYFRPGSVRLPSQTQNLYNAQGNIIAKGIYDSTTNTTTYTFTNYVDQYTNVRGSFEQVAFAKRKNATTDKTAYKMEVTLGNDTYSEEIIVDYGNKKAQPLISSTNYINNEDLSRNMTAYVNQPKNTYTKQTFVTNLTGYKFNPNAKNFKIYEVTDQNQFVDSFTPDTSKLKDVTDQFDVIYSNDNKTATVDLMKGQTSSNKQYIIQQVAYPDNSSTDNGKIDYTLDTDKTKYSWSNSYSNVNGSSTANGDQKKYNLGDYVWEDTNKDGKQDANEKGIKGVYVILKDSNGKELDRTTTDENGKYQFTGLSNGTYSVEFSTPAGYTPTTANVGTDDAV

SEQ ID NO: 8 - protein, 318 aa, Candida albicans
ARDISSTNVTDLTVSPSKIEDGGKTTVKMTFDDKNGKIQNGDMIKVAWPTSGTVKIEGYSKTVPLTVKGEQVGQAVITPDGATITFNDKVEKLSDVSGFAEFEVQGRNLTQTNTSDDKVATITSGNKSTNVTVHKSEAGTSSVFYYKTGDMLPEDTTHVRWFLNINNEKSYVSKDITIKDQIQGGQQLDLSTLNINVTGTHSNYYSGQSAITDFEKAFPGSKITVDNTKNTIDVTIPQGYGSYNSFSINYKTKITNEQQKEFVNNSQAWYQEHGKEEVNGKSFNHTVHNINANAGIEGTVKGELKVLKQDKDTKAPIA

SEQ ID NO: 9 - protein, 349 aa, Candida albicans
AKAITGIFNSIDSLTWSNAGNYAFKGPGYPTWNAVLGWSLDGTSANPGDTFILNMPCVFKFTASQKSVDLTADGVKYATCQFYSGEEFTTFSSLKCTVNNNLRSSIKALGTVTLPIAFNVGGTGSSVDLEDSKCFTAGTNTVTFNDGSKKLSIAVNFEKSTVDQSGYLTTSRFMPSLNKIATLYVAPQCENGYTSGTMGFSTSYGDVAIDCSNVHIGISKGVNDWNHPVTSESFSYTKSCSSFGISITYQNVPAGYRPFIDAYISPSDNNQYQLSYKNDYTCVDDYWQHAPFTLKWTGYKNSDAGSNGIVIVATTRTVTDSTTAVTTLPFNPSVDKTKTIEILQPIPTT

SEQ ID NO: 10 - protein, 350 aa, Candida albicans
SKEVTGVFNQFNSLIWSYTYRARYEEISTLTAKAQLEWALDGTIASPGDTFTLVMPCVYKFMTYETSVQLTANSIAYATCDFDAGEDTKSFSSLKCTVTDELTEDTSVFGSVILPIAFNVGGSGSKSTITDSKCFSSGYNTVTFFDGNNQLSTTANFLPRRELAFGLVVSQRLSMSLDTMTNFVMSTPCFMGYQSGKLGFTSNDDDFEIDCSSIHVGITNEINDWSMPVSSVPFDHTIRCTSRALYIEFKTIPAGYRPFVDAIVQIPTTEPFFVKYTNEFACVNGIYTSIPFTSFFSQPILYDEALAIGADLVRTTSTVIGSITRTTTLPFISRLQKTKTILVLEPIPTT

SEQ ID NO: 11 - protein, 338 aa, Candida albicans
ATPNVVPRKQVGNIVTAIQLTDKEGNPLGTINQYTDIYLRIEFNLPDNTVNSGDTSVITLPEELRLEKNMTFNVVDDTGTVVAIAQTDVANKTVTLTYTDYVENHANISGSLYFTSLIDFENVENESKIPIYVTVEGEKIFAGDLDYQGEGDDVNEKFSKYSWFIEDDPTEIYNVLRINPTGQTYTDLEVEDVLKTESLSYMKDTMKIERGQWTLDGNAIWQFTPEEDITDQLAVQYGPDDRNFSVHFGNIGTNEYRITYKTKIDHLPEKGETFTNYAKLTENQTVVEEVEVSRVSQTGGGEANGEQYVVEIHKEDEAGQRLAGAEFELIRNSTNQTV

SEQ ID NO: 12 - protein, 344 aa, Candida albicans
GVFDSFNSLTWSNAANYAFKGPGYPTWNAVLGWSLDGTSANPGDTFTLNMPCVFKYTTSQTSVDLTADGVKYATCQFYSGEEFTTFSTLTCTVNDALKSSIKAFGTVTLPIAFNVGGTGSSTDLEDSKCFTAGTNTVTFNDGDKDISIDVEFEKSTVDPSAYLYASRVMPSLNKVTTLFVAPQCENGYTSGTMGFSSSNGDVAIDCSNIHIGITKGLNDWNYPVSSESFSYTKTCTSNGIQIKYQNVPAGYRPFIDAYISATDVNQYTLAYTNDYTCAGSRSQSKPFTLRWTGYKNSDAGSNGIVIVATTRTVTDSTTAVTTLPFNPSVDKTKTIEILQPIPTT

SEQ ID NO: 13 - protein, 344 aa, Candida albicans
GIFNSIDSLTWSNAGNYAFKGPGYPTWNAVLGWSLDGTSANPGDTFILNMPCVFKFTASQKSVDLTADGVKYATCQFYSGEEFTTFSSLKCTVNNNLRSSIKALGTVTLPIAFNVGGTGSSVDLEDSKCFTAGTNTVTFNDGSKKLSIAVNFEKSTVDQSGYLTTSRFMPSLNKIATLYVAPQCENGYTSGTMGFSTSYGDVAIDCSNVHIGISKGVNDWNHPVTSESFSYTKSCSSFGISITYQNVPAGYRPFIDAYISPSDNNQYQLSYKNDYTCVDDYWQHAPFTLKWTGYKNSDAGSNGIVIVATTRTVTDSTTAVTTLPFNPSVDKTKTIEILQPIPTT

SEQ ID NO: 14 - protein, 344 aa, Candida albicans
GVFNSFNSLTWSNAATYNYKGPGTPTWNAVLGWSLDGTSASPGDTFTLNMPCVFKFTTSQTSVDLTAHGVKYATCQFQAGEEFMTFSTLTCTVSNTLTPSIKALGTVTLPLAFNVGGTGSSVDLEDSKCFTAGTNTVTFNDGGKKISINVDFERSNVDPKGYLTDSRVIPSLNKVSTLFVAPQCANGYTSGTMGFANTYGDVQIDCSNIHVGITKGLNDWNYPVSSESFSYTKTCSSNGIFITYKNVPAGYRPFVDAYISATDVNSYTLSYANEYTCAGGYWQRAPFTLRWTGYRNSDAGSNGIVIVATTRTVTDSTTAVTTLPFDPNRDKTKTIEILKPIPTT

SEQ ID NO: 15 - protein, 343 aa, Candida albicans
GVFNSFDSLTWTRAGNYAYKGPNRPTWNAVLGWSLDGTSANPGDTFTLNMPCVFKFITDQTSVDLTAEGVKYATCQFYSGEEFTTFSSLKCTVSNTLTSSIKALGTVTLPISFNVGGTGSSVDLESSQCFKAGTNTVTFNDGDKKISIDVDFEKTNEDASGYFIASRLIPSINKVSITYVAPQCANGYTSGAMGFIVLTGDTTIDCSNVHVGITKGLNDWNFPVSSDSLSYNKTCSSTGISITYENVPAGYRPFFDVYTSVSGQNRQLRYTNDYACVGSSLQSKPFNLRLRGYNNSEANSNGFVIVATTRTVTDSTTAVTTLPFNPSVDKTKTIEILQPIPTT

SEQ ID NO: 16 - protein, 344 aa, Candida albicans
GIFDSFNSLTWTNAASYSYRGPANPTWTAVIGWSLDGATASAGDTFTLDMPCVFKFITDQTSIDLVADGRTYATCNLNSAEEFTTFSSVSCTVTTTMTADTKAIGTVTLPFSFSVGGSGSDVDLANSQCFTAGINTVTFNDGDTSISATVDFEKSTVASSDRILLSRILPSLSQAVSLFLPQECANGYTSGTMGFSTAGTGATIDCSTVHVGISNGLNDWNYPISSESFSYTKTCTSTSVLVTYQNVPAGYRPFVDAYVSATRVSSYAMRYTNIYACVGAASVDDSFTHTWSGYSNSQAGSNGITIVVTTRTVTDSTTAVTTLPFNSESDKTKTIEILQPIPTT

SEQ ID NO: 17 - protein, 343 aa, Candida albicans
GVFNSFNSLTWANAANYGYQTPETPTWTAVLGWSLNSTTADAGDTFTLIMPCVFKFITSQTSVDLTADGVSYATCDFNAGEEFTTFSSLSCTVNSVSVSYDKASGTVKLPFSFNVGGTGSSVDLTDSKCFTAGKNTVTFTDGDTEISTSVDFQASPISSSGYIASARVVPSLNKASSLFVSPQCENGYTSGIMGFVTSQGATIDCSNINIGISKGLNDWNFPVSSESFTYTKTCSSSGIIVEYENVPAGYRPFVDAYISSENVEQYTLTYANEYTCKNGNTVVDPFTLTWTGYKNSEADSNGDIIVVTTKTVTASTTAVTTLPFNPTVDKTETIEVLQPIPTT

SEQ ID NO: 18 - protein, 346 aa, Candida albicans
GVFTSFNSLTYTNTGNYPYGGPGYPTWTAVLGWSLDGTLASPGDTFTLVMPCVFKFITTQTSVDLTANGVKYATCTFHAGEDFTTFSSMSCVVNNGLSSNIRAFGTVRLPISFNVGGTGSSVNIQDSKCFTAGTNTVTFTDGDHKISTTVNFPKTPQSSSSLVYFARVIPSLDKLSSLVVASQCTAGYASGVLGFSATKDDVTIDCSTIHVGITNGLNSWNMPVSSESFSYTKTCTPNSFIITYENVPAGYRPFIDSYVKKSATATNGFNLNYTNIYNCMDGKKGNDPLIYFWTSYTNSDAGSNGAAVVVTTRTVTDSTTAITTLPFDPTVDKTKTIEVIEPIPTT

SEQ ID NO: 19 - protein, 345 aa, Candida albicans
GVFNQFNSLIWSYTYRARYEEISTLTAKAQLEWALDGTIASPGDTFTLVMPCVYKFMTYETSVQLTANSIAYATCDFDAGEDTKSFSSLKCTVTDELTEDTSVFGSVILPIAFNVGGSGSKSTITDSKCFSSGYNTVTFFDGNNQLSTTANFLPRRELAFGLVVSQRLSMSLDTMTNFVMSTPCFMGYQSGKLGFTSNDDDFEIDCSSIHVGITNEINDWSMPVSSVPFDHTIRCTSRALYIEFKTIPAGYRPFVDAIVQIPTTEPFFVKYTNEFACVNGIYTSIPFTSFFSQPILYDEALAIGADLVRTTSTVIGSITRTTTLPFISRLQKTKTILVLEPIPTT